(19) United States
(12) Patent Application Publication
(10) Pub. No.: US 2019/0021641 A1
(43) Pub. Date: Jan. 24, 2019

(54) SYSTEM WITH COMPOSITION AND APPARATUS OF TREATING DISORDERS AND ILLNESSES AND SYSTEM FOR OBJECTIVE AND ACCURATE IDENTIFICATION, DIAGNOSIS, TRACKING, AND TREATMENT OF DISORDERS AND ILLNESSES

(71) Applicant: Seth Feuerstein, Woodbridge, CT (US)

(72) Inventor: Seth Feuerstein, Woodbridge, CT (US)

(21) Appl. No.: 16/067,277

(22) PCT Filed: Dec. 30, 2016
(86) PCT No.: PCT/US2016/069484

§ 371 Date: Jun. 29, 2018

Related U.S. Application Data
(60) Provisional application No. 62/321,574, filed on Apr. 12, 2016.
(60) Provisional application No. 62/273,947, filed on Dec. 31, 2015.

(51) Int. Cl. A61B 5/16; A61P 25/24; A61K 36/185; A61P 25/22; A61P 25/30; A61B 5/00; A61P 25/28; A61P 27/16

(52) U.S. Cl. CPC A61B 5/165; A61P 25/24; A61K 36/185; A61P 25/22; A61B 5/0002; A61B 5/4839; A61P 25/28; A61P 27/16; A61P 25/30

(57) ABSTRACT

The present disclosure provides compositions, methods, systems, and apparatuses for treating disorders or illnesses. The composition may comprise at least one substance based on or derived from cocoa beans or coffee beans provided to be used in the methods, systems, and apparatuses and consumed by a patient for treatment of a disorder or illness. The systems and apparatuses, generally used in combination with the compositions, may comprise at least one device (e.g., a sensor) configured to monitor at least one action performed by a patient, and at least one processor.

### RELATED APPLICATIONS

This application claims the benefit under 35 U.S.C. § 119(e) of U.S. Provisional Patent Application Ser. No. 62/273,947, filed on Dec. 31, 2015 and entitled “SYSTEM AND METHOD FOR OBJECTIVE AND ACCURATE IDENTIFICATION, DIAGNOSIS, TRACKING, AND TREATMENT OF MENTAL AND BEHAVIORAL ILLNESSES,” and U.S. Provisional Patent Application Ser. No. 62/321,574, filed on Apr. 12, 2016 and entitled “COMPOSITION WITH SYSTEM AND COMPUTER-READABLE MEDIUM FOR TREATING MOOD, BEHAVIORAL, AND MEDICAL DISORDERS,” each of which is hereby incorporated herein by reference in its entirety.

### BACKGROUND

Antidepressants are a well-known class of pharmaceuticals. They are generally categorized according to their mechanism of action on neurotransmitter activity (e.g., Selective Serotonin Reuptake Inhibitors; Serotonin and Norepinephrine Reuptake Inhibitors; Tricyclic antidepressants; and Monoamine Oxidase Inhibitors); with a number of drugs in each category having received marketing approval in the U.S. and elsewhere. Although widely used, antidepressants are known to have a number of drawbacks. For example, they are known to be plagued by long delays in initiating response (typically 4 or more weeks), and may have only a partial or no response.

Certain natural compounds have also been investigated for their effectiveness in treating depression. For example, substantial work has been done to study the effectiveness of various omega-3 fatty acids in treating depression (See, for example, Carlezon et al., 2005; Marangell et al., 2003; U.S. Pat. Nos. 6,852,870, 8,071,646, and 8,372,451; and U.S. Patent Publ. Nos. 2005/0267212 and 2011/0200690).

Despite this work, however, the use of omega-3 fatty acids in treating depression has failed in clinical trials (e.g., VASCEPA® (icospent ethyl), an EPA-only omega-3 fatty acid, failed in depression clinical trials), and currently no omega-3 fatty acid formulation has been approved by the U.S. Food and Drug Administration (FDA) for use in treating depression. In fact, recent meta-analyses suggest omega-3 fatty acids, on a population level, have mixed results (see, for example, Mischoulon, 2011; Appleton et al., 2010; Martins, 2009; and Young and Conquer, 2005). Compositions based on cocoa beans and coffee beans have had similarly unclear results.

Insomnia has traditionally been thought to be associated with depression. As such, certain sleep medications have been used to treat sleep difficulties associated with depression. However, not only do these medications not treat the depression itself, findings suggest that they may actually cause and/or exacerbate depression (Kripke, 2007; and Walling, 2010).

In this regard, the FDA has required the side effect “worsening of depression” to be included on the label of certain sleep medications (such as zolpidem), and this side effect known to be associated with the use of sleeping pills has been receiving increased attention in the media (see, for example, Rabin, 2012). In fact, sleep deprivation—rather than sleep inducement—has been shown to help alleviate depression (see, for example, Giedke, 2002, which teaches that sleep deprivation may help depression; See also, Giedke et al., 2003; Wirz-Justice, 1999; Adrien, 2002; Letemendia, 1986; and Wu, 1990). As such, sleep-inducing medications may be contraindicated in patients suffering from depression.

Additionally, mental, mood, behavioral, cognitive, and other disorders and illnesses are common but are often difficult to objectively and accurately identify, diagnose, track, and treat. One behavioral or cognitive disorder, obsessive compulsive disorder (OCD), may be characterized by compulsions that interfere with individuals' lives. Identification, diagnosis, and tracking of OCD has been traditionally accomplished by an interview process consisting of asking for a patient's history from the patient and from those that know the patient, such as family members. More recently, identification, diagnosis, and tracking have been considered by utilizing the patient's communications (e.g., via social media) and feedback, as done by ginger.io. Treatment has been determined based on these methodologies.

### SUMMARY

Some embodiments described herein provide compositions, combinations, and methods that overcome the problems described above and other problems, and provide a more robust treatment for disorders and illnesses, including mental, mood, behavioral, cognitive, personality, psychotic, and medical disorders and illnesses, such as depression and suicidal ideation and/or any other suitable disorder or illness, with faster onset.

Some exemplary, non-limiting embodiments are directed to a pharmaceutical composition comprising an omega-3 fatty acid formulation and a sleep-inducing agent.

In some exemplary, non-limiting embodiments, the omega-3 fatty acid formulation has greater than 90% purity.

Some exemplary, non-limiting embodiments are directed to a pharmaceutical composition comprising an omega-3 fatty acid formulation and a sleep-inducing agent, wherein the composition is substantially free of an antidepressant. According to some embodiments, a composition may be substantially free of a substance where, for example, the composition comprises (by weight, for example) less than 30%, less than 10%, less than 5%, less than 2.5%, less than 1%, less than 0.1%, less than 0.01%, or 0% of that substance.

Some exemplary, non-limiting embodiments are directed to a pharmaceutical product comprising: (a) at least one dose of an omega-3 fatty acid formulation and (b) at least one dose of a sleep-inducing agent, wherein the combination of the dose of (a) and the dose of (b) is effective for treating depression in a patient.

Some exemplary, non-limiting embodiments are directed to a pharmaceutical product comprising: (a) at least one dose of an omega-3 fatty acid formulation and (b) at least one dose of a sleep-inducing agent, wherein the combination of the doses may be effective for the treatment of depression in a patient. In some embodiments, the pharmaceutical product is packaged with instructions for administering the dose of the composition of (a) and the dose of the composition of (b).

Some exemplary, non-limiting embodiments are directed to a method of treating depression comprising administering to a patient an effective amount of a combination of an omega-3 fatty acid formulation and a sleep-inducing agent.

Some exemplary, non-limiting embodiments are directed to a method of treating depression comprising administering to a patient an effective amount of a combination of an omega-3 fatty acid formulation and a sleep-inducing agent in the absence of an antidepressant.

Some exemplary, non-limiting embodiments are directed to a method of treating depression comprising administering to a patient an effective amount of a combination of an omega-3 fatty acid formulation and a sleep-inducing agent, wherein the combination is effective for the treatment of depression even in the absence of an antidepressant.

Some exemplary, non-limiting embodiments are directed to a pharmaceutical composition comprising an antidepressant and a sleep-inducing agent.

Some exemplary, non-limiting embodiments are directed to a pharmaceutical composition comprising an antidepressant and a sleep-inducing agent that is substantially free of an omega-3 fatty acid formulation.

Some exemplary, non-limiting embodiments are directed to a system for treating at least one disorder or illness. The composition may comprise at least one substance based on or derived from cocoa beans provided to be consumed by a patient for treatment of at least one disorder or illness. The apparatus may comprise: at least one input device configured to receive at least one input from the patient; at least one processor configured to determine information to output to the patient based on the at least one input received from the patient; and at least one output device configured to output, to the patient, information assisting in treatment of the at least one disorder or illness.

In some embodiments, the at least one disorder or illness may comprise a mental or mood disorder. In some embodiments, the mental or mood disorder may comprise depression.

In some embodiments, the at least one disorder or illness may comprise a behavioral or cognitive disorder including at least one of sleep disorder, anxiety disorder, panic disorder, phobia, obsessive compulsive disorder, and substance abuse.

In some embodiments, the at least one disorder or illness may comprise a medical disorder including at least one of irritable bowel, tinnitus, chronic and acute pain, and cognitive impairment.

In some embodiments, the at least one disorder or illness may comprise a mental, mood, behavioral, cognitive, or medical disorder occurring pre-partum or post-partum.

In some embodiments, the patient may be above 65 years old.

In some embodiments, the at least one substance based on or derived from cocoa beans may comprise at least one of cocoa beans, cocoa, chocolate, flavonoids, phytochemicals, and epicatechins. In some embodiments, the composition may comprise phytochemicals. Additionally, the phytochemicals may be an anti-oxidant. Alternatively, the phytochemicals may be a non-anti-oxidant.

In some embodiments, the system may be further comprising or in combination with a dispensing device configured to dispense at least one serving of the composition to the patient.

In some embodiments, the apparatus may comprise a wearable device worn by the patient. Alternatively or additionally, the apparatus may comprise a mobile phone.

Some exemplary, non-limiting embodiments are directed to a composition and at least one computer-readable storage medium storing instructions that, when executed by at least one processor, perform a method for treating at least one disorder or illness. The method may comprise: receiving at least one input from the patient; determining information to output to the patient based on the at least one input received from the patient; and outputting, to the patient, information assisting in treatment of the at least one disorder or illness.

Some exemplary, non-limiting embodiments are directed to a system for treating at least one disorder or illness. The composition may comprise at least one substance based on or derived from coffee beans provided to be consumed by a patient for treatment of at least one disorder or illness. The apparatus may comprise: at least one input device configured to receive at least one input from the patient; at least one processor configured to determine information to output to the patient based on the at least one input received from the patient; and at least one output device configured to output, to the patient, information assisting in treatment of the at least one disorder or illness.

In some embodiments, the at least one substance based on or derived from coffee beans may comprise at least one of coffee beans, coffee grounds, coffee, filtered coffee extract, concentrated coffee extract, flavonoids, phytochemicals, and epicatechins.

Some exemplary, non-limiting embodiments are directed to a pharmaceutical product comprising: (a) at least one dose of an antidepressant and (b) at least one dose of a sleep-inducing agent, wherein the combination of the dose of (a) and the dose of (b) is effective for treating depression in a patient.

Some exemplary, non-limiting embodiments are directed to a pharmaceutical product comprising: (a) at least one dose of an antidepressant and (b) at least one dose of a sleep-inducing agent, wherein the combination of the dose of (a) and the dose of (b) is effective for treating depression in a patient, further wherein the pharmaceutical product further comprises instructions for administering each dose.

Some exemplary, non-limiting embodiments are directed to a method of treating depression comprising administering to a patient an effective amount of a combination of an antidepressant and a sleep-inducing agent.

Some exemplary, non-limiting embodiments are directed to a method of treating depression comprising administering to a patient an effective amount of a combination of an antidepressant and a sleep-inducing agent in the absence of an omega-3 fatty acid formulation.

Some exemplary, non-limiting embodiments are directed to a method of treating depression comprising administering to a patient an effective amount of a combination of an antidepressant and a sleep-inducing to a patient in need thereof, wherein the combination is effective for the treatment of depression even in the absence of an omega-3 fatty acid formulation.

In some exemplary, non-limiting embodiments, the antidepressant may be selected from the group consisting of tricyclic antidepressants, selective serotonin reuptake inhibitors (SSRI's), selective serotonin norepinephrine reuptake inhibitors, norepinephrine dopamine reuptake inhibitors, and alpha-2 antagonist/serotonin 5HT2-3 receptor antagonists. In some exemplary, non-limiting embodiments, the antidepressant may be a tricyclic antidepressant selected from the group consisting of trazodone, doxepin, amitriptyline, amoxapine, clomipramine, desipramine, doxepin, imipramine, maprotiline, nortriptyline, protriptyline, and trimipramine, provided that if the antidepressant is trazodone it may not also comprise the sleep-inducing agent.

In some exemplary, non-limiting embodiments, the tricyclic antidepressant may be selected from the group consisting of doxepin, amitriptyline, amoxapine, clomipramine, desipramine, doxepin, imipramine, maprotiline, nortriptyline, protriptyline, and trimipramine.

In some exemplary, non-limiting embodiments, the antidepressant is a selective serotonin reuptake inhibitor selected from the group consisting of citalopram, escitalopram, femoxetine, fluoxetine, fluvoxamine, paroxetine, sertraline and zimeldine.

In some exemplary, non-limiting embodiments, the antidepressant may be a selective serotonin reuptake inhibitor selected from the group consisting of duloxetine, venlafaxine, desvenlafaxine, milnacipran, and clovoxamine.

In some exemplary, non-limiting embodiments, the antidepressant may be a norepinephrine dopamine reuptake inhibitor.

In some exemplary, non-limiting embodiments, norepinephrine dopamine reuptake inhibitor antidepressant may be bupropion.

In some exemplary, non-limiting embodiments, the antidepressant may be a alpha-2 antagonist/serotonin 5HT2-3 receptor antagonist.

In some exemplary, non-limiting embodiments, the alpha-2 antagonist/serotonin 5HT2-3 receptor antagonist antidepressant may be mirtazapine.

In some exemplary, non-limiting embodiments, the sleep-inducing agent may be selected from the group consisting of antihistamines, hypnotics, trazadone tricyclic antidepressant, melatonin, melatonin receptor agonists, tryptophan, and Valerian root.

In some exemplary, non-limiting embodiments, the sleep-inducing agent may be selected from the group consisting antihistamines, hypnotics, trazadone tricyclic antidepressants, melatonin, melatonin receptor agonists, and Valerian root.

In some exemplary, non-limiting embodiments, the sleep-inducing agents may be selected from the group consisting of antihistamines, hypnotics, melatonin, melatonin receptor agonists, and Valerian root.

In some exemplary, non-limiting embodiments, the sleep-inducing agent may be selected from the group consisting of hypnotics, melatonin, melatonin receptor agonists, and Valerian root.

In some exemplary, non-limiting embodiments, the sleep-inducing agent may be selected from the group consisting of hypnotics, melatonin, and melatonin receptor agonists.

In some exemplary, non-limiting embodiments, the sleep-inducing agent may be a hypnotic.

In some exemplary, non-limiting embodiments, the hypnotics may be selected from the group consisting of benzodiazepine hypnotics and non-benzodiazepine hypnotics.

In some exemplary, non-limiting embodiments, the benzodiazepine hypnotics may be selected from the group consisting of alprazolam, brotizolam, clonazepam, cinolazepam, diazepam, estazolam, etizolam, flunitrazepam, flurazepam, loprazolam, lormetazepam, nimetazepam, qauzepam temazepam, and triazolam.

In some exemplary, non-limiting embodiments, the non-benzodiazepine hypnotics may be selected from the group consisting of imidazopyridines non-benzodiazepine hypnotics, pyrazolopyrimidines non-benzodiazepine hypnotics, cyclopyrrolones non-benzodiazepine hypnotics, and β-carboline non-benzodiazepine hypnotics.

In some exemplary, non-limiting embodiments, the imidazopyridines non-benzodiazepine hypnotics may be selected from the group consisting of zolpidem (tartrate), alpidem, necopidem, and saripidem.

In some exemplary, non-limiting embodiments, the pyrazolopyrimidines non-benzodiazepine hypnotics may be selected from the group consisting of zaleplon, divaplon, fasiplon, indiplon, lorediplon, ocinaplon, panadiplon, and taniplon.

In some exemplary, non-limiting embodiments, the cyclopyrrolones non-benzodiazepine hypnotics are selected from the group consisting of eszopiclone, zopiclone, pagoclone, pazinaclone, suproclone, and suriclone.

In some exemplary, non-limiting embodiments, the β-carboline non-benzodiazepine hypnotics may be selected from the group consisting of abecarnil and gedocarnil.

In some exemplary, non-limiting embodiments, the omega-3 fatty acid formulation may comprise eicosapentaenoic acid (EPA) and docosahexaenoic acid (DHA) in a weight to weight ratio from about 3.5:1 to about 6.99:1.

In some exemplary, non-limiting embodiments, the omega-3 fatty acid formulation may comprise EPA and DHA in a weight to weight ratio from about 4.01:1 to about 6.99:1.

In some exemplary, non-limiting embodiments, the omega-3 fatty acid formulation may comprise EPA and DHA in a weight to weight ratio from about 4.01:1 to about 5:1.

In some exemplary, non-limiting embodiments, the omega-3 fatty acid formulation may comprise EPA and DHA in a weight to weight ratio of EPA:DHA that is approximately 4.09:1.

In some exemplary, non-limiting embodiments, the omega-3 fatty acid formulation may comprise EPA and DHA, which, when taken together, may be greater than 90% of the formulation by weight.

In some exemplary, non-limiting embodiments, the omega-3 fatty acid formulation may comprise EPA and DHA, which, when taken together, may be greater than 91% of the formulation by weight.

Some exemplary, non-limiting embodiments are directed to a method of treating depression comprising administering an insomnia therapy program to a patient suffering from depression.

Some exemplary, non-limiting embodiments are directed to a method of treating depression comprising administering an insomnia therapy program to a patient suffering from depression and further comprising administration to the patient an antidepressant in an amount effective to treat depression in combination with the insomnia therapy program.

Some exemplary, non-limiting embodiments are directed to a method of treating depression comprising administering an insomnia therapy program to a patient suffering from depression and further comprising administration to the patient of an omega-3 fatty acid formulation in an amount effective to treat depression in combination with the insomnia therapy program.

Some exemplary, non-limiting embodiments are directed to a method of treating depression comprising administering an insomnia therapy program to a patient suffering from depression and further comprising administration to the patient an antidepressant in an amount effective to treat depression in combination with the insomnia therapy program in the absence of administering a omega-3 fatty acid formulation.

Some exemplary, non-limiting embodiments are directed to a method of treating depression comprising administering an insomnia therapy program to a patient suffering from depression and further comprising administration to the patient of an omega-3 fatty acid formulation in an amount effective to treat depression in combination with the insomnia therapy program in the absence of administering an antidepressant.

Some exemplary, non-limiting embodiments are directed to a method of treating depression comprising administering an insomnia therapy program to a patient suffering from depression, either alone or in combination with administration of one or more antidepressants and/or an omega-3 fatty acid formulation to the patient.

In some exemplary, non-limiting embodiments, depression may be treatment-resistant depression.

In some exemplary, non-limiting embodiments, treatment of depression or treatment-resistant depression is a 50% or greater reduction in a depression symptoms ratings scale score over the course of clinical treatment from starting point to endpoint depression symptoms rating scales.

In some exemplary, non-limiting embodiments, depression symptoms rating scales are selected from the group consisting of HRSD₁₇, QIDS-SR₁₆, and MADRS.

In some exemplary, non-limiting embodiments, the treatment of depression or treatment resistant depression comprises the depression going into remission.

In some exemplary, non-limiting embodiments, the treatment of depression or treatment-resistant depression comprises the patient achieving less than or equal to 7 on the HRSD₁₇ scale.

In some exemplary, non-limiting embodiments, the treatment of depression or treatment-resistant depression comprises the patient achieving less than or equal to 5 on the QIDS-SR₁₆.

In some exemplary, non-limiting embodiments, the treatment of depression or treatment-resistant depression comprises the patient achieving than or equal to 10 on the MADRS.

In some exemplary, non-limiting embodiments, the patient is pregnant.

In some exemplary, non-limiting embodiments, the treatment of depression or treatment-resistant depression occurs within about 8 weeks of first treatment, about 7 weeks of first treatment, about 6 weeks of first treatment, about 5 weeks of first treatment, about 4 weeks of first treatment, about 3 weeks of first treatment, about 2 weeks of first treatment, or about 1 week of first treatment.

In some exemplary, non-limiting embodiments, onset of the attenuation of depression or treatment-resistant depression occurs within about 8 weeks of first treatment, about 7 weeks of first treatment, about 6 weeks of first treatment, about 5 weeks of first treatment, about 4 weeks of first treatment, about 3 weeks of first treatment, about 2 weeks of first treatment, or about 1 week of first treatment.

Other exemplary, non-limiting embodiments provide a pharmaceutical combination comprising an omega-3 fatty acid and a sleep-inducing agent. The combination includes embodiments in which the omega-3 fatty acid and sleep-inducing agent are in separate dosage forms, but provided together, for example in a single package.

In some embodiments the purity of the omega-3 fatty acid may be greater than 90%.

Some embodiments may alternatively or additionally include a pharmaceutical combination comprising an antidepressant and a sleep-inducing agent. The antidepressant and sleep-inducing agent may be provided together, but as separate dosage forms, or may comprise a single dosage form (e.g., as a pharmaceutical formulation).

Some exemplary, non-limiting embodiments are directed to methods of treating depression comprising administering to a patient a non-benzodiazepine hypnotic.

Some exemplary, non-limiting embodiments are directed to methods of treating depression comprising administering to a patient a non-benzodiazepine hypnotic wherein a non-benzodiazepine hypnotic is selected from the group consisting of zolpidem (tartrate) and eszopiclone.

In some exemplary, non-limiting embodiments the sleep-inducing agent may be selected from trazodone, diphenhydramine, zolpidem, eszopiclone, tryptophan, and melatonin. In other embodiments the sleep-inducing agent may be a benzodiazepine.

In other exemplary, non-limiting embodiments, one or more of the above embodiments (or elements within the embodiments) are suitably combined. By way of illustration, such an embodiment could reflect the combination of the embodiment directed to “a method of treating depression comprising administering to a patient an effective amount of a combination of an omega-3 fatty acid formulation and a sleep-inducing agent” and the embodiment directed to “the treatment of depression or treatment resistant depression comprises the patient achieving less than or equal to 7 on the HRSD_{N} scale.”

In other exemplary, non-limiting embodiments, the elements comprising one or more of the embodiments herein are independent of each other such that one or more of the elements may be suitably excluded to comprise an additional embodiment thereof that is a subset of the original embodiment. By way of illustration, the embodiments recited herein defining benzodiazepine hypnotics as being “selected from the group consisting of alprazolam, brotizolam, clonazepam, cinolazepam, diazepam, estazolam, etizolam, flunitrazepam, flurazepam, loprazolam, lormetazepam, nimetazepam, qauzepam temazepam, and triazolam” also represent an embodiment to a subset or sub-combination thereof—e.g., “selected from the group consisting of alprazolam, brotizolam, and clonazepam” or any other suitable subset.

Some aspects include an apparatus for assisting treatment, diagnosis, and response to treatment of at least one disorder or illness. The apparatus may comprise: at least one sensor configured to monitor at least one action performed by a patient; and at least one processor configured to: count a number of similar actions monitored by the at least one sensor; determine whether the number of similar actions is above a threshold number within a threshold duration of time; and in response to determining that the number of similar actions is above the threshold number within the threshold duration of time: generate a response relating to at least one disorder or illness; and provide the response relating to the at least one disorder or illness to the patient via a user interface of the apparatus and/or to a device separate from the apparatus. The at least one action may comprise a repetitive motion sensed by the at least one sensor, and the at least one sensor may comprise at least one accelerometer and/or at least one gyroscope. The at least one action may comprise a change in position of the patient sensed by the at least one sensor, and the at least one sensor may comprise a positioning module, at least one accelerometer, and/or at least one gyroscope. The apparatus may comprise a wearable device worn by the patient. The apparatus may comprise a mobile phone carried by the patient. The apparatus may comprise a remote device such as an additional sensor affixed to a body part of the patient, the additional sensor being configured to communicate with the mobile phone.

Further aspects include a method for assisting treatment of at least one disorder or illness. The method may comprise: monitoring at least one action performed by a patient; counting a number of similar actions monitored; determining whether the number of similar actions is above a threshold number within a threshold duration of time; in response to determining that the number of similar actions is above the threshold number within the threshold duration of time: generating a response relating to at least one disorder or illness; and providing the response relating to the at least one disorder or illness to the patient and/or to a device. The method may comprise receiving an optional input from the patient relating to whether the similar actions undertaken are interfering with well-being of the patient.

Additional aspects include a system for assisting treatment of at least one disorder or illness. The system may comprise: an apparatus in possession of a patient; a first device separate from the apparatus; and a second device separate from the apparatus, wherein: the apparatus and/or the first device is configured to monitor at least one action performed by the patient, and the apparatus, the first device, and/or the second device is configured to: count a number of similar actions monitored; determine whether the number of similar actions is above a threshold number within a threshold duration of time; and in response to determining that the number of similar actions is above the threshold number within the threshold duration of time: generate a response relating to at least one disorder or illness; and provide the response relating to the at least one disorder or illness to the patient and/or to a supervisor associated with the patient. The first device separate from the apparatus may comprise at least one sensor configured to monitor the at least one action performed by the patient, the first device being physically unconnected to the patient. The second device separate from the apparatus may be configured to: receive first information relating to the at least one action performed by the patient and second information relating to at least one action performed by at least one additional patient; and update at least one database based on the first information and the second information. The apparatus, the first device, and/or the second device may be configured to receive an optional input from the patient relating to whether the similar actions undertaken are interfering with well-being of the patient.

The foregoing is a non-limiting summary of the application. It should be appreciated that the acts described and claimed herein may be used in other combinations, even if not expressly recited in those combinations.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a block diagram of an exemplary system with which some embodiments may operate;

FIG. 2A is a flowchart of an exemplary process that may be used in some embodiments;

FIG. 2B is a flowchart of an additional exemplary process that may be used in some embodiments; and

FIG. 3 illustrates an example of a computing system environment with which some embodiments may operate.

### DETAILED DESCRIPTION

The inventor has recognized and appreciated a need for new treatments for disorders and illnesses, including mental, mood, behavioral, cognitive, personality, psychotic, and medical disorders and illnesses, such as depression and suicidal ideation and/or any other suitable disorder or illness, and in particular for new pharmaceutical combinations to provide more robust treatments for such disorders with faster onset.

The inventor has also recognized and appreciated that these and other disorders and illnesses can be treated more effectively by using treatments with components and techniques that have shown mixed or even negative results in many cases. Embodiments of the present application may treat these disorders or illnesses using such components and techniques.

Generally speaking, and as discussed in greater detail in the illustrative and non-limiting Examples provided herein, some embodiments are directed to pharmaceutical compositions and methods for their use to treat depression. In various described exemplary, non-limiting embodiments, the pharmaceutical compositions include combinations of omega-3 fatty acids, products based on or derived from cocoa beans or coffee beans, pharmacological sleep agents, and non-pharmacological sleep therapies, and may include other ingredients such as antidepressants. For example, some exemplary, non-limiting embodiments include (1) a pharmaceutical composition comprising an omega-3 fatty acid formulation and a sleep-inducing agent; (2) a pharmaceutical composition comprising an omega-3 fatty acid formulation and a sleep-inducing agent, wherein the composition is substantially free of an antidepressant; (3) a pharmaceutical composition comprising an omega-3 fatty acid formulation and a sleep-inducing agent, wherein the composition further comprises an antidepressant; (4) a pharmaceutical composition comprising an antidepressant and a sleep-inducing agent; (5) a pharmaceutical composition comprising an antidepressant and a sleep-inducing agent, wherein the composition is substantially free of an omega-3 fatty acid formulation; (6) a pharmaceutical composition comprising an antidepressant and a sleep-inducing agent, wherein the composition further comprises an omega-3 fatty acid formulation; (7) methods of treating depression comprising administering to a patient the above examples; (8) a method of treating depression comprising administering an insomnia therapy program to a patient suffering from depression; (9) a method of treating depression comprising administering an insomnia therapy program to a patient suffering from depression further comprising administering to the patient an antidepressant in an amount effective to treat depression in combination with said insomnia therapy program; (10) a method of treating depression comprising administering an insomnia therapy program to a patient suffering from depression further comprising administration to the patient an omega-3 fatty acid formulation in an amount effective to treat depression in combination with said insomnia therapy program; and (11) a method of treating depression comprising administering an insomnia therapy program to a patient suffering from depression further comprising administration to the patient an omega-3 fatty acid formulation and an antidepressant in an amount effective to treat depression in combination with said insomnia therapy program. Some exemplary, non-limiting embodiments include (1) administering an insomnia therapy program to a patient suffering from depression; (2) a method of treating depression comprising administering an insomnia therapy program to a patient suffering from depression further comprising administration to the patient a pharmaceutical composition comprising a formulation based on or derived from cocoa beans in an amount effective to treat depression in combination with said insomnia therapy program; and (3) a method of treating depression comprising administering an insomnia therapy program to a patient suffering from depression further comprising administering to the patient a pharmaceutical composition comprising a formulation based on or derived from coffee beans in an amount effective to treat depression in combination with said insomnia therapy program.

Various definitions are provided herein, explicitly and/or through usage, and it is understood that such definitions will be applied by those of skill in the art in understanding some embodiments.

The terms “a” and “an” do not denote a limitation of quantity, but rather denote the presence of at least one of the referenced item.

An “active agent” may mean any compound, substance, element, or mixture that when administered to a patient alone or in combination with another agent confers, directly or indirectly, a physiological effect on the patient. When the active agent is a compound, salts, solvates (including hydrates) of the free compound, or salt, crystalline and non-crystalline forms, as well as various polymorphs of the compound, are included. Compounds may contain one or more asymmetric elements such as stereogenic centers, stereogenic axes and the like (e.g., asymmetric carbon atoms) so that the compounds can exist in different stereoisomeric forms. These compounds can be, for example, racemates or optically active forms. All stereoisomers, diastereomers, Z- and E-forms, in purified and mixture forms are included. Accordingly, when a compound is recited by specific name or a class of compounds is recited, all these forms are intended to be included. By illustration, active agents as provided herein include, for example, antidepressants, omega-3 fatty acid formulations, and sleep-inducing agents.

A “dosage form” may be any unit of administration (“unit dose”) of one or more active agents. As such, a “pharmaceutical composition” as used herein may be presented in the form of a dosage form or unit dose and may comprise one or more active agents. Thus, a pharmaceutical composition as used herein could, for example, provide two active agents admixed together in a unit dose or provide two active agents combined in a dosage form wherein the active agents are physically separated and/or have different release rates. Pharmaceutical compositions may include any suitable formulation including, for example, capsules, tablets, injections, and liquids and may be administered through any suitable route including oral, buccal, parenteral, intravenous, intramuscular, rectal, transdermal, and the like. Excipients used to formulate the pharmaceutical formulations may be any of those suitable for the respective dosage form such as fillers, stabilizers, extenders, binders, humidifiers, surfactants, lubricants, and the like. A “combined pharmaceutical product” as used herein is a combination of two more doses of two or more different active agents combined in separate dosage forms, which are not admixed.

“Therapeutically effective amount” and/or “effective amount” means an amount effective, when administered to a human or non-human patient, to provide any therapeutic benefit. A therapeutic benefit may be an amelioration of symptoms—e.g., an amount effective to decrease the symptoms of binge-eating disorder or a major depressive disorder. In certain circumstances a patient may not present symptoms of a condition for which the patient is being treated. Thus a therapeutically effective amount of a compound is also an amount sufficient to provide a significant positive effect on any indicia of a disease, illness, disorder, or condition (e.g., an amount sufficient to significantly reduce the frequency and severity of binge eating behavior or depressive symptoms). A significant effect on an indicia of a disorder or condition includes a statistically significant in a standard parametric test of statistical significance such as Student's T-test, where p<0.05; though the effect need not be significant in some embodiments. “Patient” as used herein means human or non-human animals.

“Based on” a substance as used herein means that a formulation or composition naturally includes the substance. “Derived from” a substance as used herein means that a formulation or composition includes something that is artificially produced to duplicate or imitate the substance.

Frequency of dosage may vary depending on the compound used and the particular type of depression treated. For most disorders and illnesses a dosage regimen of once per day is preferred. Dosage regimens in which the active agent, whether omega-3 fatty acid, cocoa- or coffee-based or derived formulation, antidepressant, or sleep-inducing agent is administered 2 times daily may occasionally be more helpful.

It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease in the patient undergoing therapy. Patients may generally be monitored for therapeutic effectiveness using assays suitable for the condition being treated or prevented, which will be familiar to those of ordinary skill in the art.

Moreover, for each of the sleep-inducing agent(s), antidepressant(s), omega-3 fatty acid formulation(s), and cocoa- or coffee-based or derived formulation(s), the dose needed for use in the inventive compositions and combinations to effectively treat depression may, in some embodiments, be lower than the dose needed to effectively treat depression when used alone (this lower dose may be referred to herein as a “suboptimal” dose, in that it is below an amount that is required for each of these ingredients to reach optimal therapeutic effect for any given patient when used alone). For example, in some embodiments, the dose of an antidepressant needed to effectively treat a patient's depression is lower when the antidepressant is used together with a non-benzodiazepine hypnotic (in a composition or combination, as taught herein) than when the antidepressant is used alone. Similarly, for each of the sleep-inducing agent(s), antidepressant(s), omega-3 fatty acid formulation(s), and cocoa- or coffee-based or derived formulation(s), the dose needed for use in combination with an insomnia therapy program to effectively treat depression may be lower than the dose needed to effectively treat depression when used alone. Thus, without being bound to any particular theory, the use of two or more of the following: sleep-inducing agent(s), antidepressant(s), omega-3 fatty acid formulation(s), and cocoa- or coffee-based or derived formulation(s), and insomnia sleep therapy program(s), in various embodiments has been found to be surprisingly effective in treating depression.

Depression includes depressive disorders listed in the American Psychiatric Association's Diagnostic and Statistical Manual of Mental Disorders (DSM-V); such as major depressive disorder, dysthymic disorder, and depressive disorder not otherwise specified (for instance, premenstrual dysphoric disorder). In some embodiments the depression is treatment-resistant depression. “Treatment-resistant depression” as used herein indicates patients who do not respond to two separate trials of different antidepressants of adequate dose and duration in the current episode.

Depression may be considered effectively treated when a patient's symptoms, as measured by a depression symptom rating scale, improve.

“Depression symptoms rating scale” refers to any one of a number of standardized questionnaires, clinical instruments, or symptom inventories utilized to measure symptoms and symptom severity in depression. Such rating scales are often used in clinical studies to define treatment outcomes, based on changes from the study's entry point(s) to endpoint(s). Such depression symptoms rating scales include, but are not limited to, The Quick Inventory of Depressive-Symptomatology Self-Report (QIDS-SR₁₆), the 17-Item Hamilton Rating Scale of Depression (HRSD₁₇), the 30-Item Inventory of Depressive Symptomatology (IDS-C₃₀), or The Montgomery-Asperg Depression Rating Scale (MADRS), and Beck Depression Inventory II. Such ratings scales may involve patient self-report or be clinician-rated. A 50% or greater reduction in a depression symptoms ratings scale score over the course of a clinical trial (starting point to endpoint) is typically considered a favorable response for most depression symptoms rating scales. “Remission” in clinical studies of depression often refers to achieving at, or below, a particular numerical rating score on a depression symptoms rating scale, i.e., less than or equal to 7 on the HRSD₁₇; or less than or equal to 5 on the QIDS-SR₁₆; or less than or equal to 10 on the MADRS, or less than or equal to 9 on the Beck Depression Inventory II. An alternative measure commonly used to assess depression and response is the Patient Health Questionnaire No. 9 (PHQ-9). A reduction in the score is generally used as a measure of improvement and the score level is used to estimate none, mild, moderate, or severe disease categories. Moving from one category to another is generally considered significant change. As such treatment of depression or treatment-resistant depression may be evidenced by an improvement to the patient progressing to next less severe PHQ-9 category after treatment which is termed “PHQ-9 categorical improvement” as used herein.

As used herein, “sleep-inducing compounds” and/or “sleep-inducing agents” include the following: (1) antihistamines such as BENADRYL® (diphenhydramine); (2) “hypnotics” which include (a) benzodiazepines such as alprazolam, brotizolam, clonazepam, cinolazepam, diazepam, estazolam, etizolam, flunitrazepam, flurazepam, loprazolam, lormetazepam, nimetazepam, qauzepam temazepam, and HALCION® (triazolam), (b) “non-benzodiazepine hypnotics” also known as Z-drugs such as (i) “imidazopyridines” including AMBIEN (CR)® zolpidem (tartrate), alpidem, necopidem, and saripidem, (ii) “pyrazolopyrimidines” such as zaleplon, divaplon, fasiplon, indiplon, lorediplon, ocinaplon, panadiplon, and taniplon, (iii) “cyclopyrrolones” such as LUNESTA® (eszopiclone), IMOVANE® (zopiclone), pagoclone, pazinaclone, suproclone, and suriclone, and (iv) β-carbolines such as abecarnil, gedocarnil; (3) certain tricyclic antidepressants including DESYREL® (trazodone); (4) melatonin and melatonin receptor agonists such as ramelteon; and (5) other sleep-inducing agents such as tryptophan and Valerian root and melatonin.

The term “antidepressant” as used herein includes (1) tricyclic antidepressants including DESYREL® (trazodone), doxepin, amitriptyline, amoxapine, clomipramine, desipramine, doxepin, imipramine, maprotiline, nortriptyline, protriptyline, and trimipramine; (2) selective serotonin reuptake inhibitors (SSRI's) including citalopram, escitalopram, femoxetine, fluoxetine, fluvoxamine, paroxetine, sertraline, and zimeldine; selective serotonin norepinephrine reuptake inhibitors including duloxetine, venlafaxine, desvenlafaxine, milnacipran, and clovoxamine; (3) norepinephrine dopamine reuptake inhibitors such as bupropion; and (4) alpha-2 antagonist/serotonin 5HT2-3 receptor antagonists such as mirtazapine.

Wherein embodiments comprise both an antidepressant and a sleep-inducing agent, the antidepressant and the sleep-inducing agent are different compounds. For example, trazadone is known in the art both as an antidepressant and as a sleep-inducing agent. Accordingly, an embodiment that is directed to a pharmaceutical composition including both an antidepressant and a sleep-inducing agent may include trazadone as either the antidepressant or as the sleep-inducing agent, but not both.

As used herein, an “omega-3 fatty acid formulation” includes EPA and/or DHA. More particularly, an omega-3 fatty acid formulation according to some embodiments may comprise EPA and DHA in a weight to weight ratio from about 3.5:1 to about 6.99 to 1, from about 4.01:1 to about 6.99:1, or from about 4.01:1 to about 5:1. Some embodiments also provide a highly purified omega-3 fatty acid formulation in which the weight to weight ratio of EPA:DHA is approximately 4.09:1. The EPA and DHA may be present in the formulation in either the triglyceride form or in the form of esterified fatty acid. Capsules typically contain the ethyl esters forms of EPA and DHA. Candy formulations typically contain the triglyceride forms of EPA and DHA.

Some embodiments also provide highly purified omega-3 fatty acid formulations in which the content of EPA and DHA, taken together, is at least 70%, at least 75%, at least 84%, or at least 85% of the formulation by weight, and the omega-3 fatty acids comprise at least 85%, at least 90%, at least 91%, at least 95%, at least 98%, or at least 99% of the formulation by weight. Additionally, some embodiments provide omega-3 fatty acid formulations in which the amount of cholesterol in the formulation is less than 5% by weight, less than 2.5% by weight, or less than 1% by weight. Some embodiments also include omega-3 fatty acid formulations in which the formulation comprises less than 20 milliequivalents per kg peroxides, less than 10 milliequivalents per kg peroxides, or less than 5 milliequivalents per kg peroxides. See also U.S. Pat. No. 8,071,646, incorporated in its entirety by reference herein.

As discussed herein, some embodiments are directed to methods of treating depression by administering an insomnia therapy program to a patient suffering from depression (alone or in combination with one or more antidepressants, an omega-3 fatty acid formulation, and/or a cocoa- or coffee-based or derived formulation).

Cocoa-based or derived formulation(s) may include at least one of cocoa beans, cocoa, chocolate, flavonoids, phytochemicals, and epicatechins. In some embodiments, the composition may comprise phytochemicals. Additionally, the phytochemicals may be anti-oxidant. Alternatively, the phytochemicals may be non-anti-oxidant.

Coffee-based or derived formulation(s) may include at least one of coffee beans, coffee, coffee grounds, filtered coffee extract, concentrated coffee extract, flavonoids, phytochemicals, and epicatechins. In some embodiments, the composition may comprise phytochemicals. Additionally, the phytochemicals may be anti-oxidant. Alternatively, the phytochemicals may be non-anti-oxidant.

As used herein, the term “insomnia therapy program” refers to a non-pharmacological, computer-implemented cognitive behavioral therapy program useful for treating insomnia in a patient. The terms “insomnia therapy program,” “insomnia talk therapy program,” and “talk therapy program for insomnia” may be used interchangeably herein.

One example of an insomnia therapy program that may be used in some embodiments includes the program described in Vincent and Lewycky, 2009 and Vincent et al., 2009, the entirety of each of which is hereby incorporated by reference. While such programs have been shown to be effective for treating insomnia (See, for example, Vincent, 2009), they have not been used to treat depression.

In one embodiment, an insomnia therapy program used in some embodiments includes a plurality of software modules with which the patient interacts over a network. The software modules are stored in a computer system which includes non-transitory computer readable medium for storing the software, and one or more processors for executing instructions contained in the software. The software modules may be web-based such that the patient interacts with the software modules over the Internet.

As set forth in Table 1, each software module is intended to affect a cognitive and/or behavioral change in the patient.

TABLE 1
Insomnia Therapy Program: Software Modules

| | |
|---|---|
| Module 1 | Includes psychoeducation about insomnia (e.g., information about |
| | normal sleep, types of sleep disorders). |
| | Presents the cognitive behavioral model of insomnia (Morin, 1993). |
| | Patient is instructed to avoid clock-watching to reduce hyperarousal in |
| | the bedroom. |
| Module 2 | Includes information regarding sleep hygiene (e.g., implication of |
| | daytime napping for sleep, information regarding effects of alcohol |
| | consumption on sleep) and stimulus control (e.g., encouragement to |
| | avoid engendering arousal in the bedroom environment, removing of |
| | oneself from bed if unable to sleep, going to bed only when sleepy). |
| | Patient is instructed to choose two habits to change. |
| Module 3 | Presents relaxation training and provides digital audio files (e.g., |
| | MP3s) for paced breathing, progressive muscle relaxation, imagery- |
| | induced relaxation, and self-hypnosis. Patient is instructed to practice |
| | relaxation strategies on daily basis, as well as to continue practicing |
| | sleep hygiene and stimulus control. |
| | Patient is asked to choose the relaxation exercises that they most liked |
| | and to practice with those. |
| | There is no demand to work on all 4 relaxation exercises concurrently. |
| Module 4 | Teaches sleep restriction (Spielman et al., 1987) and discusses how to |
| | gradually taper off hypnotic medications only under the direction of a |
| | physician. |
| | Patient is against tapering if they had comorbid medical conditions as a |
| | safety precaution. |
| | For SRT, patient is informed about how to calculate a sleep window |
| | but is discouraged from using this strategy if currently sleeping less |
| | than 4 hours per night. |
| Module 5 | Cognitive therapy, including instruction and modeling regarding the |
| | identification and correction of automatic thoughts that may increase |
| | arousal (Morin, 1993); instruction regarding scheduled problem |
| | solving (Dugas, 2003); and instruction and modeling regarding the |
| | downward arrow technique (Burns, 1980). |
| | Patient has the opportunity to listen to audio files of cognitive therapy |
| | between actors portraying patients with insomnia and the first author |
| | acting as cognitive therapist. |
| | Patient is instructed to monitor thoughts and attempt to replace anxiety- |
| | provoking thoughts with more realistic alternatives. |

The discussion herein and the following Examples set forth and illustrate various exemplary embodiments, which are understood to be illustrative and non-limiting.

### Example 1

### Case Study 1: 38 Year Old Male

A 38 year old male patient seen for depression had taken a serotonin specific reuptake inhibitor, fluoxetine for four weeks without improvement. The patient was started on 20 mg daily for one month and this was increased to a maximum dose of 60 mg daily over the subsequent two months. The higher doses improved depression minimally—Beck Depression Inventory II score changed from 28 to 26 over three months of fluoxetine monotherapy.

Subsequently, addition of AMBIEN® at only 2.5 mg (one half of a 5 mg tablet) led to improvement in depression rating scales of 38% in 4 days after addition to the medical regimen. In this case, the depression was rated with the Beck Depression Inventory II and at first visit the patient's score was 28 and at the upper end of the moderate range.

Over the course of the patient's treatment with fluoxetine as monotherapy, the patient's mood remained depressed with mild improvement to a Beck Depression Inventory II score of 26 after dose increases. When the AMBIEN® was added, the score improved an additional 8 points to 18, and the score for sleep was unchanged. At the time of this treatment course, the patient was also taking albuterol inhaler as needed for asthma and had no other active medical conditions or medications.

### Example 2

### Case Study 2: 44 Year Old Female

A 44 year old female patient presented with depression that had been unresponsive to three known antidepressants over the preceding year (fluoxetine; venlafaxine; and the tricyclic amitryptiline). A new trial of fluoxetine at 20 mg was started concurrently with LUNESTA® (dose: 2 mg at bedtime). Mood returned to normal/mild depression range based on PHQ-9 score. This showed efficacy because the PHQ-9 score before treatment initiation was 17 (indicating “moderately severe depression”) and had dropped to 7 (indicating “mild depression”) in six days at subsequent visit. This showed a shift of two categories in the PHQ-9 scale. On the PHQ the patient rated their sleep problems as “not at all” when asked about trouble falling or staying asleep, or sleeping too much. Antidepressant effects are known to occur in 2-6 weeks when used as previously studied.

During the course of treatment this patient was utilizing birth control via NUVARING® monthly and was also prescribed alprazolam as needed for anxiety. Use of alprazolam was limited to plane flights and attending crowded social events.

### Example 3

### Case Study 3: 41 Year Old Male

A 41 year old male patient presented with no complaints of sleep difficulties but with complaints of depression that had been unresponsive to fluoxetine and citalopram. An omega-3 fatty acid formulation was started at 2 grams daily composed of a >90% pure omega-3 fatty acid formulation with a ratio of EPA:DHA of approximately 4:1 with AMBIEN® (5 mg at bedtime) and in 5 days mood showed significant improvement by patient report (patient reported no longer feeling depressed).

Questioning the patient evidenced no difficulty sleeping—either too much or too little—before the AMBIEN® was started. There remained no sleep complaints throughout the treatment course—either of which can occur with depression. During the course of treatment patient was also taking atorvastatin, 10 mg daily.

The above Examples surprisingly show and suggest inter alia that non-benzodiazepine hypnotics (which are known in the art to exacerbate depression) are effective in treating depression (including treatment-resistant depression) when combined with antidepressant(s) or omega-3 fatty acid(s).

### Example 4

### Case Study 4: 62 Year Old Male

A 62 year old male patient suffering from depression had failed trials of several medications including fluoxetine, citalopram, trazodone. In addition, the patient had taken ATIVAN® (lorazepam) on rare occasions related to anxiety inducing situations which also did not improve the patient's depression.

The patient was started on a trial of fluoxetine and ATIVAN® (lorazepam) regularly at bedtime (0.5 mg and an additional 0.5 mg as needed for dose of 1.0 mg) (Riemann, 2009) and the patient's mood improved, with rating scale improvements (PHQ-9 score from 22 to 10) of greater than 50% in one week of treatment.

This example surprisingly shows and suggests, among other things, that benzodiazepine hypnotics (which have been shown in the art to have no significant effect in treating depression) may be effective in treating depression (including treatment-resistant depression) when combined with antidepressant(s).

### Example 5

### Case Study 5: 35 Year Old Female

A 35 year old female patient with onset of depression in pregnancy was interested in natural remedies. The patient was started on an omega-3 fatty acid (OMAX-3®) with minimal effect after 4 weeks: PHQ-9 score went from 17 to 16.

Two weeks later the patient's PHQ-9 remained at 16. The patient was not complaining of sleep difficulties, in fact describing increased sleep. Despite this, Valerian (obtained from GNC®)—which is known to help induce sleep (Gyllenhaal, 2000)—was added to the regimen (of OMAX-3®) at 500 mg one hour before bed. The patient described a desired bed time of ten PM and dose was taken at 9 PM.

One week later the patient described improved mood and the patient's PHQ-9 score improved to 11 despite the fact Valerian is a central nervous system depressant and is believed to cause mild depression (Houghton, 1999).

The above Example surprisingly shows and suggests, among other things, that sleep-inducing agents such as Valerian (which have not been shown in the art to be effective in treating depression) may be effective in treating depression (including treatment-resistant depression) when combined with omega-3 fatty acid(s).

### Example 6

### Case Study 6: 36 Year Old Female

Onset of depression in pregnancy is common. In this 36 year old pregnant female patient who presented with new onset of depression, the patient's PHQ-9 score was 15 and the patient detailed no sleep complaints. The patient was concerned about taking any pharmaceutical and was initiated on an omega-3 fatty acid with high purity (>90% omega-3 and a ratio of EPA to DHA of approximately 4:1, sold as OMAX-3®) and simultaneously started on an insomnia therapy program as described herein that provided cognitive behavioral therapy for insomnia over six weeks, despite a lack of insomnia complaints.

Specifically, the insomnia therapy program was organized into modules, and was offered as an internet-based application as a mix of interactive multimedia content, an interactive sleep diary, sleep restriction, and relaxation training. Module 1 included psychoeducation about insomnia (e.g., information about normal sleep and/or types of sleep disorders) and presented the cognitive behavioral model of insomnia. Module 2 included information regarding sleep hygiene (e.g., implication of daytime napping for sleep and/or information regarding effects of alcohol consumption on sleep) and stimulus control (e.g., encouragement to avoid engendering arousal in the bedroom environment, removing of oneself from bed if unable to sleep, and/or going to bed only when sleepy). Module 3 presented relaxation, passive muscle relaxation, imagery-induced relaxation, and self-hypnosis. Participants were asked to choose the relaxation exercises that they most liked and to practice with those. There was no demand to work on all four relaxation exercises concurrently. Module 4 introduced the concept of sleep restriction. Module 5 introduced cognitive therapy, including correction of automatic thoughts that may increase arousal, instruction regarding scheduled problem solving, and instruction and modeling regarding the downward arrow technique. Module 6 were exercises in mindfulness meditation.

The patient's depression improved after 3 weeks to a PHQ-9 score of 4.

The above Example surprisingly shows and suggests, among other things, that insomnia therapy programs when combined with omega-3 fatty acid(s) (which have not been shown in clinical trials to be effective in treating depression when administered independently) may be effective in treating depression (including treatment-resistant depression) when co-administered.

### Example 7

### Case Study 7: Insomnia Therapy Program to Treat Depression in a Male Patient

A male patient with a history of depression presented and described a history of trying several antidepressants without success. The patient's PHQ-9 was 16 and the patient endorsed 9 hours of sleep, and rated the PHQ-9 sleep question at 0. The patient did, however, describe sleeping about 4 hours during the day and 5 hours at night. The patient was given a regimen of sleep hygiene and sleep restriction from an interactive insomnia therapy program as described herein.

Specifically, the program was a six module program which also tracked sleep metrics in a sleep diary. The modules focused on insomnia and goal setting, relaxation exercises, sleep restriction, mindfulness, cognitive restructuring, and sleep hygiene. The patient continued to sleep 9 hours, but this shifted to all being at night as a result of the intervention. The patient's PHQ-9 score dropped to 4.

The above Example surprisingly shows and suggests, among other things, that insomnia therapy programs (which have not been shown in the art to be effective in treating depression) are effective in treating depression (including treatment-resistant depression).

### Example 8

### Case Study 8: 41 Year Old Female

A 41 year old woman, who was feeling depressed and in treatment, was not recovering. She was provided with a software program providing sections to improve sleep (which was not her primary complaint) as well as told to add foods that included fat and sugar in a more thoughtful way. She had removed chocolate from her diet in an effort to lose weight. She used the software program, which included sleep hygiene exercises and tracked her sleep, and included a cognitive behavioral therapy element and began ingesting chocolate. Within 3 weeks her mood improved significantly.

The above Example surprisingly shows and suggests, among other things, that insomnia therapy programs when combined with chocolate (which have not been shown in clinical trials to be effective in treating depression when administered independently) may be effective in treating depression (including treatment-resistant depression) when co-administered.

### Example 9

### Case Study 9: 40 Year Old Male

A 40 year old male, who had been experiencing anxiety and depression for many months and failed cognitive behavioral therapy, began to go through an insomnia cognitive behavioral therapy digital program and had been to a chocolate-producing region (Nicaragua). He began to consume chocolate from his trip at the same time and his mood and anxiety were completely improved.

The above Example surprisingly shows and suggests, among other things, that insomnia therapy programs when combined with chocolate (which have not been shown in clinical trials to be effective in treating depression when administered independently) may be effective in treating depression (including treatment-resistant depression) when co-administered.

### Example 10

### Case Study 10: 45 Year Old Female

A 45 year old female was in good health but suffered from irritable bowel syndrome. She had tried diet change and prescription medications without success. She then decided to take an omega-3 fatty acid and utilize software-based programming for anxiety management and sleep improvement. This combination was successful in treating her irritable bowel syndrome.

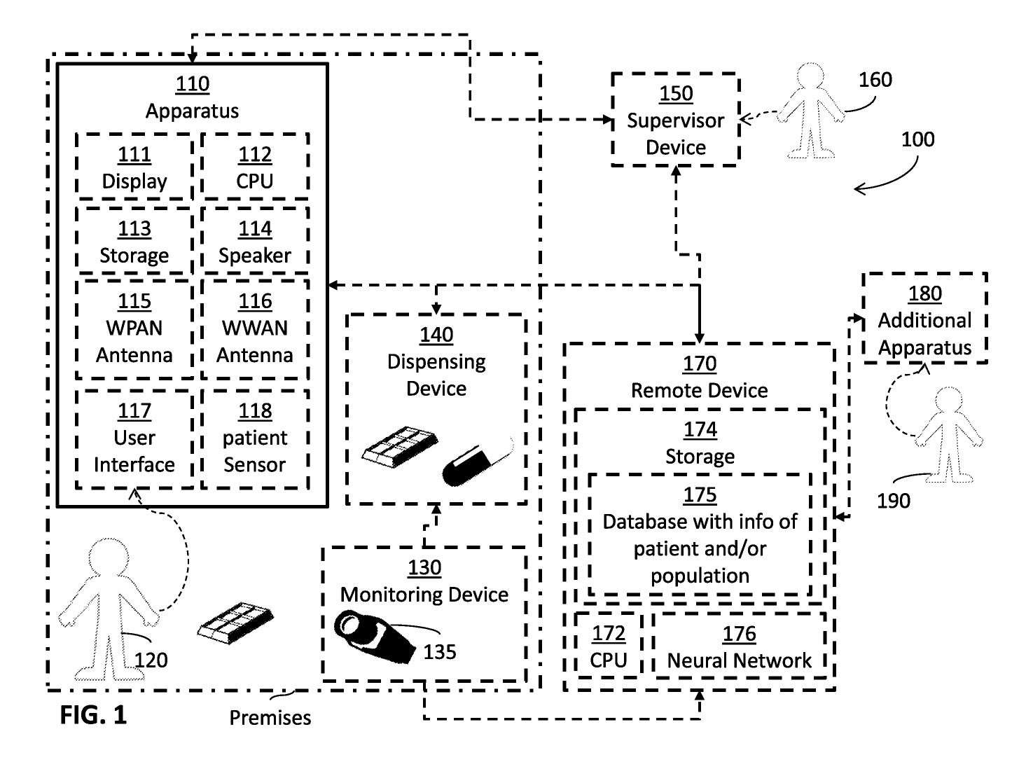

The above Example surprisingly shows and suggests, among other things, that insomnia therapy programs when combined with omega-3 fatty acids (which have not been shown in clinical trials to be effective in treating irritable bowel syndrome when administered independently) may be effective in treating irritable bowel syndrome when co-administered. Referring initially to FIG. 1, a block diagram of an exemplary system 100 with which some embodiments may operate is illustrated therein. According to some embodiments, system 100 may be for treating at least one disorder or illness and may include apparatus 110. Apparatus 110 may be a desktop or laptop computer or tablet, a mobile device in the possession of a patient 120 (e.g., carried by the patient 120 in the patient's 120 pocket), carried or worn by the patient 120, such as a smartphone, a smart watch, a fitness tracker, sensor-equipped clothing or accessories (such as glasses), or any other suitable device, although embodiments are not limited to operating with any particular type of device. Additionally, apparatus 110 may include a display 111, a CPU 112, storage 113, a global positioning system (GPS) module (not shown), a speaker 114, a WPAN antenna 115 (e.g., a Bluetooth Low Energy, Z-Wave, or any other suitable antenna), a wireless wide area network (WWAN) antenna 116 (e.g., a cellular network antenna), a user interface 117, and/or a patient sensor 118. Alternatively or additionally, apparatus 110 may include purpose-built hardware, purpose-designed software executing on existing hardware, and/or existing hardware and software providing information that may be used as described below. In some embodiments, purpose-built hardware may provide more data and more specific data than existing hardware.

According to some embodiments, the patient 120 may operate the apparatus 110 through the user interface 117. Additionally, the user interface 117 may include a graphical user interface (GUI), a speech- or audio-controlled interface, a collection of buttons or other tactile mechanisms, a touch screen or pad, and/or any other suitable interface, as embodiments are not limited in this respect. The apparatus 110 may receive input from a patient 120 through the user interface 117. For example, apparatus 110 may display or speak the available treatment modules, and patient 120 may select which module to execute. Additionally, the patient 120 may input personal information such as height, weight, personal preferences (such as what kind of notifications to receive and how often), and/or any other suitable user entry.

According to some embodiments, the patient sensor 118 may include at least one accelerometer, at least one gyroscope, a positioning module (such as a local positioning system module), a camera, a heat sensor, a microphone, a motion detector, and/or any other suitable sensor. Alternatively or additionally, the patient sensor 118 may measure characteristics (such as biometric characteristics) relating to the patient 120, including but not limited to when the patient 120 wears the apparatus 110.

According to some embodiments, system 100 may include a dispensing device 140. The dispensing device 140 may receive information relating to treatment of the at least one disorder or illness from the apparatus 110. For example, the information received by dispensing device 140 may include a selection from the patient 120, such as a choice between compositions like a cocoa product or a coffee product. Alternatively, the information received by dispensing device 140 may include a prescription for patient 120, such as pills or other medication a patient would normally obtain from a pharmacy. One example of medication is d-cycloserine. For example, dispensing device 140 may dispense (e.g., like a vending machine) chocolate, coffee products, or d-cycloserine or other learning enhancers, omega-3 fatty acids, and/or mood stabilizers to the patient 120 before a software module runs on apparatus 110 to prime the effectiveness of the module.

Alternatively or additionally, dispensing device 140 may include a delivery device for the patient to take the composition. For example, the delivery device may include at least one of a cup, a pill, a liquid suspension, and a nasal spray.

Alternatively or additionally, patient 120 may take a medication or composition independently of dispensing device 140, as shown in FIG. 1. For example, patient 120 may pick up the composition from a pharmacy or receive it in the mail and take it.

According to some embodiments, the composition may comprise at least one substance based on or derived from cocoa beans provided to be consumed by a patient for treatment of at least one disorder or illness. Alternatively, the composition may comprise at least one substance based on or derived from coffee beans provided to be consumed by a patient for treatment of at least one disorder or illness. In some embodiments, the at least one disorder or illness may comprise a mood disorder. In some embodiments, the mood disorder may comprise depression.

According to some embodiments, apparatus 110 may recommend the composition to patient 120 based on contextual information. Contextual information may include the time of day, the location, and/or other information available to apparatus 110. For example, chocolate may be recommended in the evening, while coffee may be recommend in the morning. In some embodiments, the recommendation or other processes described herein may be based on history of the patient 120 (such as patient preferences or health information) or de novo.

In some embodiments, the at least one substance based on or derived from cocoa beans may comprise at least one of cocoa beans, cocoa, chocolate, flavonoids, phytochemicals, and epicatechins. In some embodiments, the composition may comprise phytochemicals. Additionally, the phytochemicals may be anti-oxidant. Alternatively, the phytochemicals may be non-anti-oxidant.

In some embodiments, the at least one substance based on or derived from coffee beans may comprise at least one of coffee beans, coffee grounds, coffee, filtered coffee extract, concentrated coffee extract, flavonoids, phytochemicals, and epicatechins.

In some embodiments, the at least one disorder or illness may comprise a mental, behavioral, or cognitive disorder including at least one of sleep disorder, anxiety disorder, panic disorder, phobia, obsessive compulsive disorder, and substance abuse. Alternatively or additionally, the at least one disorder or illness may comprise a medical disorder including at least one of irritable bowel, tinnitus, chronic and acute pain, and cognitive impairment. Cognitive impairment may include any worsening of cognitive function, which may be anything less than normal cognitive function, including mild cognitive impairment or more significant worsening of cognitive function.

In some embodiments, the at least one disorder or illness may be occurring pre-partum or post-partum. In some embodiments, patient 120 may be above 65 years old, and the at least one disorder or illness may comprise a mood or behavioral disorder.

According to some embodiments, system 100 may alternatively or additionally be for assisting behavioral or cognitive illness treatment and may include apparatus 110.

According to some embodiments, apparatus 110 may be given only to patients 120 that have shown some indication or belief of potentially suffering from a disorder or illness (like a mental, behavioral, or cognitive disorder). Alternatively, apparatus 110 may be available to available to any person for use.

According to some embodiments, the patient sensor 118 may detect, recognize, and/or measure at least one action performed by the patient 120, including but not limited to when the patient 120 wears the apparatus 110. In some embodiments, the at least one action may include a motion of the patient 120 (e.g., a turn of a doorknob or a handwashing), a change in position of the patient 120 or lack thereof, an utterance or noise from the patient 120, and/or any other suitable action. Additionally, the at least one action may indicate at least one disorder or illness, like a mental, mood, behavioral, cognitive, or medical disorder or illness, such as repetitive turning of a doorknob caused by OCD or frequent utterances of profanity caused by Tourette syndrome. Alternatively or additionally, the at least one action may indicate a neurodegenerative disorder or a movement disorder of unknown origins, such as jerky movements caused by Huntington's disease or essential tremor, or any other medical disorder or illness.

According to some embodiments, the at least one action may be detected, recognized, and/or measured in real-time, over 24-hour periods, or at any other suitable timing or interval. Additionally, apparatus 110 may generate a report relating to the at least one action, which may be stored and/or transmitted to other devices, such as supervisor device 160 or remote device 170, which are described below. Moreover, apparatus 110 may generate a report of changes to earlier reports, which may indicate the response of the patient 120 to treatment and/or worsening (e.g., relapse) or improvement of the progression or improvement.

In some embodiments, the at least one action may include a biological or biometric change or indication sensed from the patient 120. For example, the at least one action may include a change or indication of heart rate, blood pressure, body temperature, brain activity, and/or any other characteristic of the patient 120.

According to some embodiments, apparatus 110 may assess the medical state (e.g., the neurological condition) of the patient 120 based on the at least one action. For example, apparatus 110 may interpret the at least one action to assess the medical state of the patient 120. Additionally, apparatus 110 may identify, diagnose, track, and treat at least one disorder or illness (e.g., a mental, mood, behavioral, cognitive, or medical illness) based on the at least one action. For example, apparatus 110 may determine whether the patient 120 is suffering from a disorder or illness based on whether readings relating to the patient 120 are outside a normal range. Moreover, apparatus 110 may assess the severity of the disorder or illness. Alternatively or additionally, apparatus 110 may assess the response of the patient 120 to different interventions or treatments, which will be described below.

According to some embodiments, the assessments and determinations described herein may be based on contextual information. Contextual information may include the time of day of the at least one action, the location in which the at least one action is performed, and/or other information relating to the at least one action. In some embodiments, the assessments and determinations described herein may be based on history of the patient 120 or de novo.

According to some embodiments, apparatus 110 (via the CPU 112 or another component, for example) may count a number of similar actions monitored by the patient sensor 118. An action may be considered similar to another action based on a signature of the action. For example, a first particular movement of the patient's 120 wrist (such as in turning a doorknob) may generate a pattern of readings in the patient sensor 118 that may be compared to other movements. This pattern of readings may be considered a signature for that particular movement. If a second movement is sufficiently similar to that signature, the apparatus 110 may conclude that the second movement is similar to the first movement. The inventor has recognized and appreciated that disorders and illnesses of repeated behavior may allow either simple devices (such as a fitness tracker or pedometer) or complex devices (like a smartwatch) to collect data.

According to some embodiments, apparatus 110 may create a profile for the patient 120 based on the actions monitored and the signatures recorded. Additionally, this profile may include the signatures, which may be verified by the patient 120 or another as discussed above, and/or which apparatus 110 may verify independently. This profile may be stored on apparatus 110 and/or transmitted to another device, including monitoring device 130, treatment device 140, supervisor device 150, and/or remote device 170, which are discussed below. The profile may allow identification, diagnosis, tracking, and/or treatment to be customized for the patient 120.

According to some embodiments, the apparatus 110 may determine whether the number of similar actions is above a threshold number (and/or outside a normal range) within a threshold duration of time. For example, a single turning of a doorknob within five hours may not indicate that the patient 120 is suffering from OCD, while one hundred turns of a doorknob may indicate the opposite. As another example, a patient's 120 lack of movement significantly away from an object such as a sink for one minute may not indicate any disorder or illness, while the same lack of movement for multiple hours may indicate the opposite. Actions that are outside normal ranges such as these may provide objective physical markers upon which identification, diagnosis, tracking, and treatment can be determined and linked. Identification, diagnosis, tracking, and treatment may be linked by their shared basis on these objective physical markers, such as the at least one action.

According to some embodiments, objective physical markers such as those described above may provide new or previously observed signs that a supervisor, clinician, and/or the apparatus 110 may use to identify, diagnose, track, and/or treat a disorder or illness. For example, any observed action of the patient may serve as such a sign.

Additionally, apparatus 110 may, in response to determining that the number of similar actions is above the threshold number within the threshold duration of time, generate a response relating to a disorder or illness and provide the response relating to the disorder or illness to the patient 120 via user interface 117 of the apparatus 110 and/or to a device separate from the apparatus. For example, the apparatus 110 may generate a data point for use in making an ultimate diagnosis or generate an actual diagnosis itself. Alternatively or additionally, the apparatus 110 may generate a message asking for verification (e.g., from the patient 120) of the nature of the similar actions monitored, notifying the patient 120 and/or a supervisor of the similar actions (which may include transmitting any number of signs as discussed above), and/or intervening to help prevent the patient 120 from performing the actions. For instance, if a patient 120 is spending an increased amount of time engaged in repetitive motion, which may indicate worsening of symptoms of OCD or another disorder or illness, the apparatus 110 can notify the patient 120 and/or a clinician. In addition, the apparatus 110 can inform the patient 120 of his or her status and engage him or her in exposure and response prevention exercises, such as activities that help the patient 120 focus on something else.

According to some embodiments, the interventions provided may adapt in real-time based on present conditions. For example, if the patient 120 is not responding to a particular intervention, such as a video or activity, the apparatus 110 and/or another device (as discussed below) may switch to a different intervention, such as a mild medication.

According to some embodiments, system 100 may also include monitoring device 130. Alternatively, apparatus 110 may operate independently. Monitoring device 130 may be separate from the apparatus 110 and physically unconnected to the patient 120, while still being in the same premises, vicinity, room, or structure as the apparatus 110 and the patient 120. Additionally, monitoring device 130 may include at least one sensor 135 that monitors at least one action performed by the patient 120. The at least one sensor 135 may include a motion detector, a camera, a heat sensor, a microphone, and/or any other suitable sensor. For example, monitoring device 130 may include a security system or other monitoring system located in rooms (e.g., in corners of each room) in which the patient 120 spends time, such as rooms of the patient's 120 residence or the patient's 120 hospital room. The inventor has recognized and appreciated that using monitoring devices in places in which the patient 120 is comfortable and/or already spending time may be far less expensive and more likely to capture typical activity than pre-established and/or robotic environments or “cages,” as has been used to monitor patients and animals previously. Additionally, monitoring device 130 may monitor the behavior of the patient 120 as an alternative to or in addition to the monitoring performed by apparatus 110.

According to some embodiments, monitoring device 130 may transmit information relating to at least one action performed by the patient 120 (which may include signs) to another device for recognition and processing of the information. For example, monitoring device 130 may capture at least one action of the patient 120, such as recording occurrences of a repetitive motion, and transmit such recordings to a device such as remote device 170 discussed below.

According to some embodiments, monitoring device 130 may even process the information relating to at least one action performed by the patient 120 in order to recognize the at least one action. For example, monitoring device 130 may detect, recognize, and/or measure the at least one action, such as is described above with relation to apparatus 110. In some embodiments, monitoring device 130 may perform any or all of the functions relating to processing of the at least one action (including but not limited to counting a number of similar actions, determining whether the number is outside a normal range, generating a response, and providing the response to the patient 120 and/or to a supervisor associated with the patient 120) as an alternative to or in addition to that performed by apparatus 110.

According to some embodiments, dispensing device 140 may alternatively or additionally be a treatment device 140. The treatment device 140 may receive information relating to treatment of the disorder or illness from the apparatus 110, the monitoring device 130, a remote device 170 (discussed below), and/or a supervisor device 150. For example, the information received by treatment device 140 may include a prescription for the patient 120, such as pills or other medication a patient would normally obtain from a pharmacy. One example of medication is d-cycloserine. Additionally, treatment device 140 may provide the interventions discussed above, which may come in the form of the medication prescribed (which treatment device 140 may dispense to the patient 120), a video or other multimedia for the patient 120, activities in which the patient 120 is asked to participate, and/or any other suitable interventions. For example, treatment device 140 may dispense (e.g., like a vending machine) d-cycloserine or other learning enhancers, omega-3 fatty acids, and/or mood stabilizers to the patient 120 shortly before providing each of multiple activities to prime the effectiveness of the activities.

In some embodiments, supervisor device 150 may be operated by a supervisor 160 of the patient 120, which may be a clinician, a physician, a physician assistant, a nurse, a parent or guardian, a spouse, a caretaker, and/or any other suitable person. Alternatively or additionally, the supervisor device 150 may be an autonomous device not needing direct or continuous human interaction. Additionally, supervisor device 150 may be in a remote location relative to the apparatus 110 and the patient 120, although supervisor device 150 may also be in the same premises, room, or structure as the apparatus 110.

According to some embodiments, supervisor device 150 may process the information relating to at least one action (which may include signs, as discussed above) performed by the patient 120 in order to recognize the at least one action. For example, supervisor device 150 may detect, recognize, and/or measure the at least one action, such as is described above with relation to apparatus 110. In some embodiments, supervisor device 150 may perform any or all of the functions relating to processing of the at least one action (including but not limited to counting a number of similar actions, determining whether the number is outside a normal range, generating a response, and providing the response to the patient 120 and/or to a supervisor associated with the patient 120) as an alternative to or in addition to that performed by apparatus 110.

According to some embodiments, system 100 may further include a remote device 170 that is separate from the apparatus 110. For example, remote device 170 may be in a remote location relative to the apparatus 110, although remote device 170 may also be in the same premises, room, or structure as the apparatus 110. Remote device 170 may include at least one CPU 172 and at least one storage 174, which may store at least one database 175. Additionally, remote device 170 may receive information relating to at least one action performed by the patient 120 from the apparatus 110 and/or the monitoring device 130.

According to some embodiments, remote device 170 may process the information relating to at least one action performed by the patient 120 in order to recognize the at least one action. For example, remote device 170 may detect, recognize, and/or measure the at least one action, such as is described above with relation to apparatus 110. In some embodiments, remote device 170 may perform any or all of the functions relating to processing of the at least one action (including but not limited to counting a number of similar actions, determining whether the number is outside a normal range, generating a response, and providing the response to the patient 120 and/or to a supervisor associated with the patient 120) as an alternative to or in addition to that performed by apparatus 110.

According to some embodiments, system 100 may also include one or more additional apparati 180 corresponding to the apparatus 110 for additional patients 190. The remote device 170 may also receive information relating to at least one action performed by the additional patients 190 from the additional apparati 180 and/or corresponding monitoring devices (not shown). Additionally, the remote device 170 may update the at least one database 175 based on the first information and the second information. For example, the remote device 170 may receive information about the behavior of patient 120 and information about the behavior of patient 190 and store the information in the database 175.

According to some embodiments, remote device 170 may be or include an artificial intelligence system (for example, neural network 176) that can acquire information about patients, examine the information, and offer interventions in a single closed system or an open, modular system. Additionally, the remote device 170 may learn (for example, using neural network 176) from the information about multiple patients, which may provide more and more training data that the remote device 170 can use to increase the accuracy and effectiveness of identification, diagnosis, tracking, and treatment of disorders and illnesses. For example, remote device 170 may collect signatures of actions and enter them into the at least one database 175 in association with potential diagnoses and treatments. Over time, remote device 170 may learn and improve the available knowledge relating to OCD and/or other disorders and illnesses. Additionally, remote device 170 may categorize these disorder and illnesses into types and sub-types that have not been previously described, as well as improve the ability to predict which treatments are most effective for each type and sub-type. In some embodiments, remote device 170 may define new signatures for these disorders and illnesses, types, and sub-types. Alternatively or additionally, remote device 170 may develop new diagnostic and tracking algorithms.

The inventor has recognized and appreciated that the devices and/or techniques described herein may be used in relation to any disorder or illness, and may be suitable for a depression and suicide monitoring system. For example, the inventor has recognized and appreciated that little attention has been paid to the physical activity often associated with depression and suicidal tendencies, as the focus has been on mental processes or lack of behavior. Although signatures may not yet exist for depression and other conditions that may lead to suicide, the inventor has recognized and appreciated that such signatures exist at least for enough patients to be worth investigating, and that patients need not be in an enclosed system to develop such signatures anymore. According to some embodiments, the devices and/or techniques described herein may be used to monitor for signatures indicating suicidal ideation, determine probabilities of suicide attempts, and detect suicide attempts in their earliest stages, some of which could be prevented by early detection. For example, system 100 may provide suggestions, media content, medication, or any other suitable response to a patient 120 when a suicide attempt is determined to be likely or even occurring, and may alternatively or additionally, alert a supervisor device 150 in such situations.

According to some embodiments, remote device 170 may receive and/or include genetic information, which remote device 170 may use to further improve the available knowledge, identification, diagnosis, tracking, and treatment for both populations and for particular individuals. Remote device 170 may also receive and/or include (such as in database 175) clinical information for both populations and for particular individuals, such as that stored in an electronic medical record (EMR), laboratory data, and/or pharmacy data, including medications, vital signs, general history, and other information. For example, signatures may be stored at a population level, and identification, diagnosis, tracking, and treatment may be performed based on comparison between readings about a patient 120 and the population-wide signatures. In addition, population data can be examined alone or with newly collected behavioral or cognitive activity data to develop signatures indicative of suicide risk. Additionally, information relating to patient 120 may be associated with an account belonging to or associated with patient 120.

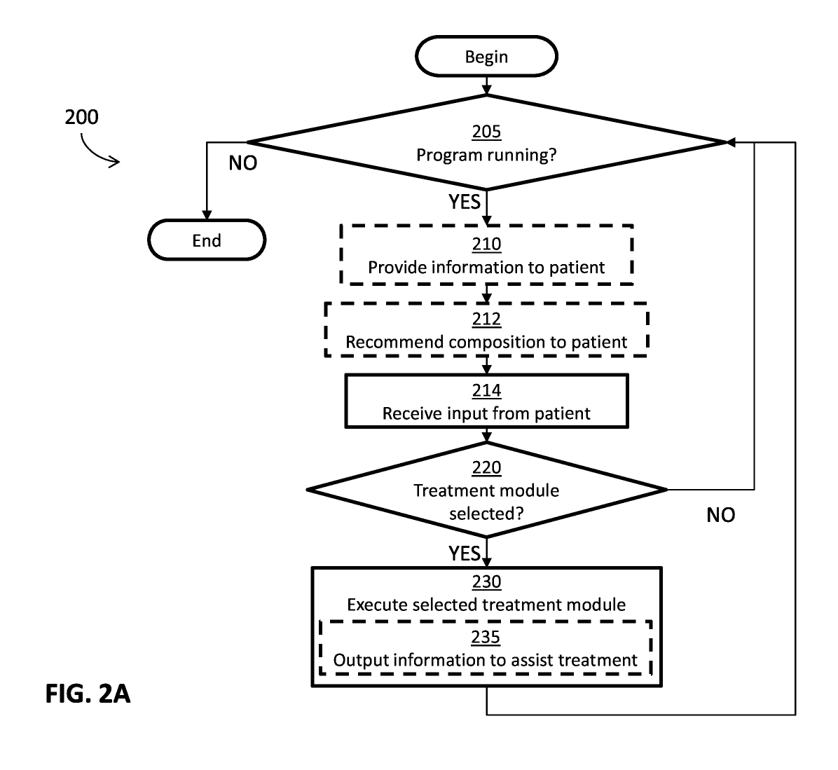

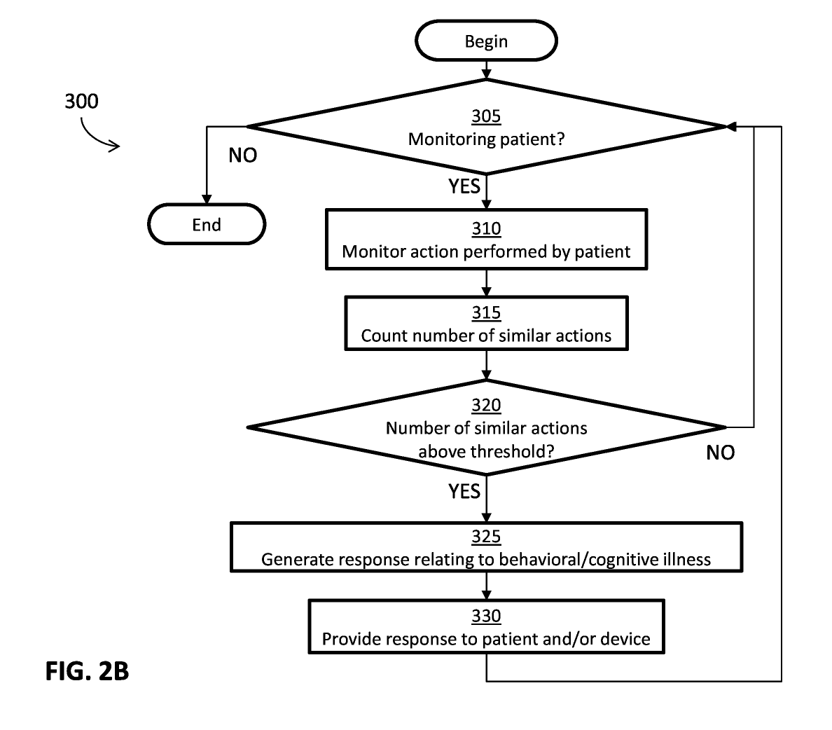

FIG. 2A illustrates a flowchart of an exemplary process 200 that may be used in some embodiments. Before the process 200 begins, a patient 120 may install on a device (which may correspond to apparatus 110 described above) and configure an application through which embodiments of the present application may operate. The process 200 begins at stage 205. At stage 205, it is determined whether the program is running. For example, if apparatus 110 is in sleep mode, the program may be in the background and not actively running. Alternatively or additionally, the patient or another party may turn off or close the program for various reasons, thereby determining when or if the process 200 may end. If the program is not running, the process 200 may end. If the program is running, the process 200 may optionally proceed to stage 210.

At stage 210, information may be provided to the patient (such as patient 120). For example, apparatus 110 may display or speak a list of insomnia treatment modules, as described above. At stage 212, a recommendation of what composition to take may be provided to the patient. For example, apparatus 110 may recommend chocolate based on the time of day or patient preferences, as described above.

At stage 220, it may be determined whether a treatment module was selected by the input from the patient. For example, it may be determined whether the patient selected a relaxation training module, as described above. If no treatment module was selected (for example, if the patient chose to not respond or close the program), the process 200 may return to stage 210, as described above. If a treatment module was selected, the process 200 may proceed to stage 230. At stage 230, the selected treatment module may be executed, as described above. Module execution may include outputting information to the patient to treat insomnia and thereby treat the disorder or illness, as described above. The process 200 may then return to stage 205.

FIG. 2B illustrates a flowchart of an exemplary process 300 that may be used in some embodiments. Before the process 300 begins, a patient 120 may install on a device (which may correspond to apparatus 110 described above) and configure an application through which embodiments of the present application may operate. The process 300 begins at stage 305. At stage 305, it is determined whether a patient (e.g., the patient 120) is being monitored. For example, the patient or another party may turn monitoring on or off for various reasons, thereby determining when or if the process 300 may end. If the patient is not being monitored, the process 300 may end. If the patient is being monitored, the process 300 may proceed to stage 310. At stage 310, at least one action performed by a patient (such as patient 120) may be monitored, as described above.

At stage 315, the at least one action may be measured. For example, a number of similar actions performed by the patient may be counted, as described above. At stage 320, it may be determined whether the at least one action is outside a normal range. For example, it may be determined whether the number of similar actions is above a threshold number, as described above. If the number of similar actions is not above the threshold number, the process 300 may return to stage 310, as described above. If the number of similar actions is above the threshold number, the process 300 may proceed to stage 325. At stage 325, a response relating to a disorder or illness may be generated, as described above. At stage 330, the response may be provided to the patient and/or a device, as described above (e.g., a notification to the patient 120 and/or supervisor device 150). The process 300 may then return to stage 305.

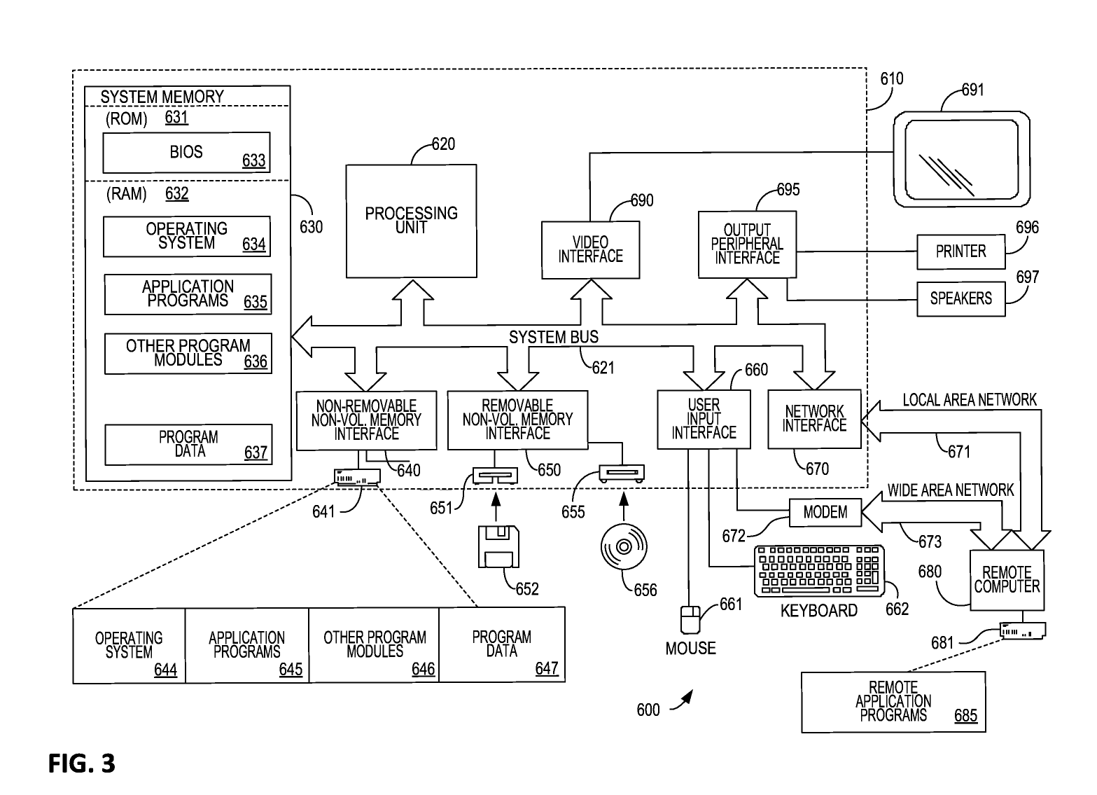

FIG. 3 illustrates an example of a suitable computing system environment 600 on which some embodiments may operate. The computing system environment 600 is only one example of a suitable computing environment and is not intended to suggest any limitation as to the scope of use or functionality of the application. Neither should the computing environment 600 be interpreted as having any dependency or requirement relating to any one or combination of components illustrated in the exemplary operating environment 600.

Embodiments of the application are operational with numerous other general purpose or special purpose computing system environments or configurations. Examples of well-known computing systems, environments, and/or configurations that may be suitable include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The application may also be practiced in distributed computing environments where tasks are performed by remote processing devices that are linked through a communications network. In a distributed computing environment, program modules may be located in both local and remote computer storage media including memory storage devices.

With reference to FIG. 3, an exemplary system for implementing embodiments of the application includes a general purpose computing device in the form of a computer 610. Computer 610, with programming or other modification to perform functions as described herein may be used to implement an apparatus for treating at least one disorder or illness or a system for objective and accurate identification, diagnosis, tracking, and treatment of disorders or illnesses. Alternatively or additionally, device 610, rather than being dedicated to a particular task, may be a computer that would, in normal operation, store or retrieve information from a storage device. In that scenario, computer 610 may be a patient's 120 computer programmed to treat at least one disorder or illness using techniques as described herein or to identify, diagnose, track, and treat disorders or illnesses using techniques as described herein.

Components of computer 610 may include, but are not limited to, a processing unit 620, a system memory 630, and a system bus 621 that couples various system components including the system memory to the processing unit 620. The system bus 621 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 610 typically includes a variety of computer readable media. Computer readable media can be any available media that can be accessed by computer 610 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 610. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 630 includes computer storage media in the form of volatile and/or nonvolatile memory such as read only memory (ROM) 631 and random access memory (RAM) 632. A basic input/output system 633 (BIOS), containing the basic routines that help to transfer information between elements within computer 610, such as during start-up, is typically stored in ROM 631. RAM 632 typically contains data and/or program modules that are immediately accessible to and/or presently being operated on by processing unit 620. By way of example, and not limitation, FIG. 3 illustrates operating system 634, application programs 635, other program modules 636, and program data 637.

The computer 610 may also include other removable/non-removable, volatile/nonvolatile computer storage media. By way of example only, FIG. 3 illustrates a hard disk drive 641 that reads from or writes to non-removable, nonvolatile magnetic media, a magnetic disk drive 651 that reads from or writes to a removable, nonvolatile magnetic disk 652, and an optical disk drive 655 that reads from or writes to a removable, nonvolatile optical disk 656 such as a CD-ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 641 is typically connected to the system bus 621 through an non-removable memory interface such as interface 640, and magnetic disk drive 651 and optical disk drive 655 are typically connected to the system bus 621 by a removable memory interface, such as interface 650.

The drives and their associated computer storage media discussed above and illustrated in FIG. 3, provide storage of computer readable instructions, data structures, program modules and other data for the computer 610. In FIG. 3, for example, hard disk drive 641 is illustrated as storing operating system 644, application programs 645, other program modules 646, and program data 647. Note that these components can either be the same as or different from operating system 634, application programs 635, other program modules 636, and program data 637. Operating system 644, application programs 645, other program modules 646, and program data 647 are given different numbers here to illustrate that, at a minimum, they are different copies. A patient 120 may enter commands and information into the computer 610 through input devices such as a keyboard 662 and pointing device 661, commonly referred to as a mouse, trackball, or touch pad. Other input devices (not shown) may include a microphone, joystick, game pad, satellite dish, scanner, or the like. These and other input devices are often connected to the processing unit 620 through a user input interface 660 that is coupled to the system bus, but may be connected by other interface and bus structures, such as a parallel port, game port or a universal serial bus (USB). A monitor 691 or other type of display device is also connected to the system bus 621 via an interface, such as a video interface 690. In addition to the monitor, computers may also include other peripheral output devices such as speakers 697 and printer 696, which may be connected through a output peripheral interface 695.

The computer 610 may operate in a networked environment using logical connections to one or more remote computers, such as a remote computer 680. The remote computer 680 may be a personal computer, a server, a router, a network PC, a peer device or other common network node, and typically includes many or all of the elements described above relative to the computer 610, although only a memory storage device 681 has been illustrated in FIG. 3. The logical connections depicted in FIG. 3 include a local area network (LAN) 671 and a wide area network (WAN) 673, but may also include other networks. Such networking environments are commonplace in offices, enterprise-wide computer networks, intranets and the Internet.

When used in a LAN networking environment, the computer 610 is connected to the LAN 671 through a network interface or adapter 670. When used in a WAN networking environment, the computer 610 typically includes a modem 672 or other means for establishing communications over the WAN 673, such as the Internet. The modem 672, which may be internal or external, may be connected to the system bus 621 via the user input interface 660, or other appropriate mechanism. In a networked environment, program modules depicted relative to the computer 610, or portions thereof, may be stored in the remote memory storage device. By way of example, and not limitation, FIG. 3 illustrates remote application programs 685 as residing on memory device 681. It will be appreciated that the network connections shown are exemplary and other means of establishing a communications link between the computers may be used.

Having thus described several aspects of at least one embodiment of this application, it is to be appreciated that various alterations, modifications, and improvements will readily occur to those skilled in the art.

Such alterations, modifications, and improvements are intended to be part of this disclosure, and are intended to be within the spirit and scope of the application. Further, though advantages of the present application are indicated, it should be appreciated that not every embodiment will include every described advantage. Some embodiments may not implement any features described as advantageous herein and in some instances. Accordingly, the foregoing description and drawings are by way of example only.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the application may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the application discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present application as discussed above. As used herein, the term “computer-readable storage medium” encompasses only a computer-readable medium that can be considered to be a manufacture (i.e., article of manufacture) or a machine. Alternatively or additionally, the application may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present application as discussed above. Additionally, it should be appreciated that according to one aspect of this embodiment, one or more computer programs that when executed perform methods of the present application need not reside on a single computer or processor, but may be distributed in a modular fashion amongst a number of different computers or processors to implement various aspects of the present application.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

Various aspects of the present application may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the application may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### REFERENCES

Adrien, J. “Neurobiological bases for the relation between sleep and depression.” Sleep Med. Rev. 6(5):341-51 (October 2002).
Appleton, K. M., Rogers, P. J. and Andrew, R. N. “Updated systematic review and meta-analysis of the effects of n-3 long-chain polyunsaturated fatty acids on depressed mood.” Am. J. Clin. Nutr. 91(31):757-770 (2010).
Burns, D. D. Feeling Good: The Mood Therapy Book. New York: New American Library (1980).
Carlezon Jr., W. A., Mague, S. D., Parow, A. M., Stoll, A. L., Cohen, B. M. and Renshaw, P. F. “Antidepressant-like effects of uridine and omega-3 fatty acids are potentiated by combined treatment in rats.” Biol. Psychiatry. 57(4):343-50 (Feb. 15, 2005).
Dugas, M. J., Ladouceur, R., Leger, E., Freeston, M. H., Langlois, F., Provencher, M. D. and Boisvert, J. M. “Group cognitive-behavioral therapy for generalized anxiety disorder: treatment outcome and long-term follow-up.” J. Consult. Clin. Psychol. 71(4):821-5 (August 2003)
Giedke H. “Therapeutic use of sleep deprivation in depression.” Sleep Med Rev. 6(5):361-77 (October 2002).
Giedke, H., Klingberg, S., Schwarzler, F. and Schweinsberg, M. “Direct comparison of total sleep deprivation and late partial sleep deprivation in the treatment of major depression.” J. Affect. Disord. 76(1-3):85-93 (September 2003).
Gyllenhaal, C., Merritt, S. L., Peterson, S. D., Block, K. I. and Gochenour, T. “Efficacy and safety of herbal stimulants and sedatives in sleep disorders.” Sleep Med Rev. 4(3):229-251 (June 2000).
Houghton, P. J. “The scientific basis for the reputed activity of Valerian.” J. Pharm. Pharmacol. 51(5):505-12 (May 1999).
Kripke, D. “Greater incidence of depression with hypnotic use than with placebo.” BMC Psychiatry 7:42 (2007).
Letemendia, F. J. “Diagnostic applications of sleep deprivation.” Can. J. Psychiatry. 31(8):731-6 (November 1986).
Marangell, L. B., Martinez, J. M., Zboyan, H. A., Kertz, B., Kim, H. F. and Puryear, L. J. “A double-blind, placebo-controlled study of the omega-3 fatty acid docosahexaenoic acid in the treatment of major depression.” Am. J. Psychiatry. 160(5):996-8 (May 2003).
Martins, J. G. “EPA but not DHA appears to be responsible for the efficacy of omega-3 long chain polyunsaturated fatty acid supplementation in depression: evidence from a meta-analysis of randomized controlled trials.” J. Am. Coll. Nutr. 28(5):525-542 (2009).
Mischoulon, D. “The impact of omega-3 fatty acids on depressive disorders and suicidality: can we reconcile 2 studies with seemingly contradictory results?” J. Clin. Psychiatry. 72(12):1574-6 (December 2011).
Morin, C. M. Insomnia: Psychological Assessment and Management. New York: Guilford Press (1993).
Rabin, R. “New Worries About Sleeping Pills.” The New York Times (Mar. 12, 2012) (available online at: http://well.blogs.nytimes.com/2012/03/12/new-worries-about-sleeping-pills).
Riemann, D. and Perlis, M. L. “The treatments of chronic insomnia: a review of benzodiazepine receptor agonists and psychological and behavioral therapies.” Sleep Med Rev. 13(3):205-14 (June 2009).
Spielman, A. J., Saskin, P. and Thorpy M. J. “Treatment of chronic insomnia by restriction of time in bed.” Sleep. 10(1):45-56 (February 1987).
U.S. Pat. No. 6,852,870.
U.S. Pat. No. 8,071,646.
U.S. Pat. No. 8,372,451.
United States Patent Publ. No. 2005/0267212.
United States Patent Publ. No. 2011/0200690.
Vincent, N. and Lewycky, S. “Logging on for better sleep: RCT of the effectiveness of online treatment for insomnia.” Sleep. 32(6):807-15 (June 2009).
Vincent, N., Lewycky, S., Hart Swain, K. and Holmqvist, M. “Logging on for Nodding Off: Empowering Individuals to Improve Their Sleep.” The Behavior Therapist. 32(6):123-126 (September 2009).
Walling, J. “Eszopiclone improves Menopausal Symptoms.” Am. Fam. Physician. 82(5):528-531 (Sep. 1, 2010).
Wirz-Justice, A. “Sleep deprivation in depression: what do we know, where do we go?” Biol. Psychiatry. 46(4):445-53 (Aug. 15, 1999).
Wu, J. C. “The biological basis of an antidepressant response to sleep deprivation and relapse: review and hypothesis.” Am. J. Psychiatry. 147(1):14-21 (January 1990).
Young, G. and Conquer, J. “Omega-3 fatty acids and neuropsychiatric disorders.” Reprod. Nutr. Dev. 45(1):1-28 (2005).

Some embodiments may be in other forms or carried out in other ways without departing from the spirit or essential characteristics thereof. The present disclosure is therefore to be considered as in all aspects illustrated and not restrictive, the scope of coverage being indicated by the appended Claims, and all changes which come within the meaning and range of equivalency are intended to be embraced therein.

Various references are cited throughout the Specification and provided in a list of references above, each of which is incorporated herein by reference in its entirety. The citation of references herein shall not be construed as an admission that such is prior art.

1. A system of treating at least one disorder or illness in a patient, comprising:
(a) a composition comprising a least one edible substance based on or derived from cocoa beans; and
(b) an apparatus comprising:
(i) at least one input device configured to receive at least one input from a patient,
(ii) at least one processor configured to determine information to output to the patient based on the at least one input received from the patient, and
(iii) at least one output device configured to output to the patient information assisting in treatment of the at least one disorder or illness.

2. The system of claim 1, wherein:
the at least one disorder or illness is depression.

3. The system of claim 1, wherein:
the at least one disorder or illness is selected from sleep disorders, anxiety disorders, panic disorders, obsessive compulsive disorders, and substance abuses.

4. The system of claim 1, wherein:
the at least one disorder or illness is irritable bowel, tinnitus, chronic and acute pain, and/or cognitive impairment.

5. The system of claim 1, wherein:
the at least one disorder or illness is occurring pre-partum, post-partum, or when the patient is above 65 years old.

6. The system of claim 1, wherein:
the at least one substance based on or derived from cocoa beans comprises at least one of cocoa beans, cocoa, chocolate, flavonoids, phytochemicals, and epicatechins.

7. The system of claim 6, wherein:
the composition comprises phytochemicals.

8. The system of claim 7, wherein:
the phytochemicals are anti-oxidant.

9. The system of claim 7, wherein:
the phytochemicals are non-anti-oxidant.

10. The system of claim 1, further comprising a dispensing device configured to dispense at least one serving of the composition to the patient.

11. The system of claim 1, wherein:
the at least one substance based on or derived from cocoa beans comprises at least one of coffee beans, coffee grounds, coffee, filtered coffee extract, concentrated coffee extract, flavonoids, phytochemicals, and epicatechins.

12. The system of claim 1, further comprising:
a delivery device including at least one of a cup, a pill, a liquid suspension, and a nasal spray.

13. An apparatus for assisting treatment of at least one disorder or illness, the apparatus comprising:
at least one sensor configured to monitor at least one action performed by a patient; and
at least one processor configured to:
count a number of similar actions monitored by the at least one sensor;
determine whether the number of similar actions is above a threshold number within a threshold duration of time; and
in response to determining that the number of similar actions is above the threshold number within the threshold duration of time:
generate a response relating to at least one disorder or illness; and
provide the response relating to the at least one disorder or illness to the patient via a user interface of the apparatus and/or to a device separate from the apparatus.

14. The apparatus of claim 13, wherein the at least one action comprises a motion sensed by the at least one sensor, and the at least one sensor comprises at least one accelerometer and/or at least one gyroscope.

15. The apparatus of claim 13, wherein the at least one action comprises a change in position of the patient sensed by the at least one sensor, and the at least one sensor comprises a positioning module, at least one accelerometer, and/or at least one gyroscope.

16. The apparatus of claim 13, wherein the apparatus comprises a wearable device worn by the patient or a mobile phone carried by the patient.

17. A system for assisting treatment of at least one disorder or illness, the system comprising:
an apparatus in possession of a patient;
a first device separate from the apparatus; and
a second device separate from the apparatus,
wherein:
the apparatus and/or the first device is configured to monitor at least one action performed by the patient, and
the apparatus, the first device, and/or the second device is configured to:
count a number of similar actions monitored;
determine whether the number of similar actions is above a threshold number within a threshold duration of time; and
in response to determining that the number of similar actions is above the threshold number within the threshold duration of time:
generate a response relating to at least one disorder or illness; and
provide the response relating to the at least one disorder or illness to the patient and/or to a supervisor associated with the patient.

18. The system of claim 17, wherein the first device separate from the apparatus comprises at least one sensor configured to monitor the at least one action performed by the patient, the first device being physically unconnected to the patient.

19. The system of claim 17, wherein the second device separate from the apparatus is configured to:
receive first information relating to the at least one action performed by the patient and second information relating to at least one action performed by at least one additional patient; and
update at least one database based on the first information and the second information.

20. The system of claim 17, wherein the system is configured to:
identify, diagnose, track, and/or treat the illness or disorder.
